(19) United States
(12) Patent Application Publication
(10) Pub. No.: US 2023/0301703 A1
(43) Pub. Date: Sep. 28, 2023

(54) Electrosurgical Tools, Electrosurgical Electrodes, and Methods of Making an Electrode for an Electrosurgical Tool

(71) Applicant: Stryker European Operations Limited, Cork (IE)

(72) Inventors: Kevin Buckley, Cork (IE); Stephen Faul, Cork (IE); Paul Sheridan, Wexford (IE)

(21) Appl. No.: 17/619,513

(22) PCT Filed: Dec. 18, 2020
(86) PCT No.: PCT/IB2020/001063

§ 371 Date: Dec. 15, 2021

Related U.S. Application Data
(60) Provisional application No. 62/949,926, filed on Dec. 18, 2019.

(51) Int. Cl. A61B 18/14

(52) U.S. Cl. CPC A61B 18/14; A61B 2018/00601; A61B 2018/1412

(57) ABSTRACT

In an example, an electrosurgical electrode for an electrosurgical tool can include a proximal end configured to receive electrosurgical energy from an electrosurgical tool and a distal end opposite the proximal end. The electrosurgical electrode can also include a cutting-electrode portion extending from the proximal end to the distal end. The cutting-electrode portion is configured for cutting tissue using the electrosurgical energy received from the electrosurgical tool. Additionally, the electrosurgical electrode can include a coagulating-electrode portion extending from the proximal end to the distal end. The coagulating-electrode portion is configured for coagulating tissue using the electrosurgical energy received from the electrosurgical tool. The electrosurgical electrode can further include an insulator between the cutting-electrode portion and the coagulating-electrode portion.

### CROSS-REFERENCE TO RELATED APPLICATIONS

This application claims the benefit of U.S. Provisional Pat. Application No. 62/949,926, filed on Dec. 18, 2019, the contents of which are hereby incorporated by reference in their entirety.

### FIELD

The present disclosure generally relates to methods and apparatus for conveying electrical energy and, more specifically, to electrosurgical tools and methods that can activate select portions of an electrosurgical electrode according to a selected mode of operation.

### BACKGROUND

Electrosurgery involves applying a radio frequency (RF) electric current (also referred to as electrosurgical energy) to biological tissue to cut, coagulate, or modify the biological tissue during an electrosurgical procedure. Specifically, an electrosurgical generator generates and provides the electric current to an active electrode, which applies the electric current (and, thus, electrical power) to the tissue. The electric current passes through the tissue and returns to the generator via a return electrode (also referred to as a “dispersive electrode”). As the electric current passes through the tissue, an impedance of the tissue converts a portion of the electric current into thermal energy (e.g., via the principles of resistive heating), which increases a temperature of the tissue and induces modifications to the tissue (e.g., cutting, coagulating, ablating, and/or sealing the tissue).

### BRIEF DESCRIPTION OF THE FIGURES

The novel features believed characteristic of the illustrative examples are set forth in the appended claims. The illustrative examples, however, as well as a preferred mode of use, further objectives and descriptions thereof, will best be understood by reference to the following detailed description of an illustrative example of the present disclosure when read in conjunction with the accompanying drawings, wherein:

FIG. 1 depicts a simplified block diagram of an electrosurgical system, according to an example.

FIG. 2 depicts a perspective view of an electrosurgical tool, according to an example.

FIG. 3A depicts a perspective view of an electrosurgical electrode, according to an example.

FIG. 3B depicts an exploded view of the electrosurgical electrode shown in FIG. 3A, according to an example.

FIG. 3C depicts a cross-sectional view of the electrosurgical electrode shown in FIG. 3A, according to an example.

FIG. 4 depicts a schematic circuit diagram of the electrosurgical system of FIG. 1, according to an example.

FIG. 5 depicts a schematic circuit diagram of the electrosurgical system of FIG. 1, according to another example.

FIG. 6 depicts a schematic circuit diagram of the electrosurgical system of FIG. 1, according to another example.

FIG. 7 depicts a flowchart for a process of making an electrosurgical electrode for an electrosurgical tool, according to an example.

### DETAILED DESCRIPTION

Disclosed examples will now be described more fully hereinafter with reference to the accompanying drawings, in which some, but not all of the disclosed examples are shown. Indeed, several different examples may be described and should not be construed as limited to the examples set forth herein. Rather, these examples are described so that this disclosure will be thorough and complete and will fully convey the scope of the disclosure to those skilled in the art.

By the term “approximately” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

As noted above, during an electrosurgical procedure, an electrosurgical generator generates and provides electrosurgical energy to an electrosurgical electrode, which applies the electrosurgical energy (and, thus, electrical power) to a patient’s tissue. In general, the electrosurgical generator modifies the power and/or waveform of the electrosurgical energy supplied to the electrosurgical tool to operate the electrosurgical tool in different modes of operation.

In conventional electrosurgical systems, the electrosurgical energy is conducted through an entirety of the electrosurgical electrode in all modes of operation. The present disclosure provides for electrosurgical systems, tools, electrodes, and methods that can additionally enhance characteristics of the electrosurgical energy applied to the patient’s tissue by selectively applying the electrosurgical energy to different portions of the electrosurgical electrode based on the mode of operation in which the electrosurgical tool is operated. Within examples, the different portions of the electrosurgical electrode can have a plurality of different sizes and/or a plurality of different shapes that can help to enhance one or more properties of the electrosurgical energy applied to the target tissue. In this way, the electrosurgical electrode can enhance and/or improve operational performance of the electrosurgical tool relative to conventional electrosurgical tools that conduct the electrosurgical energy through an entirety of the electrosurgical electrode for all modes of operation.

In an example, an electrosurgical electrode for an electrosurgical tool can include a proximal end configured to receive electrosurgical energy from an electrosurgical tool and a distal end opposite the proximal end. The electrosurgical electrode can also include a cutting-electrode portion extending from the proximal end to the distal end. The cutting-electrode portion is configured for cutting tissue using the electrosurgical energy received from the electrosurgical tool. Additionally, the electrosurgical electrode can include a coagulating-electrode portion extending from the proximal end to the distal end. The coagulating-electrode portion is configured for coagulating tissue using the electrosurgical energy received from the electrosurgical tool. The electrosurgical electrode can further include an insulator between the cutting-electrode portion and the coagulating-electrode portion.

In another example, an electrosurgical system includes an electrosurgical electrode and an electrosurgical tool. The electrosurgical electrode includes a proximal end configured to receive electrosurgical energy from an electrosurgical tool and a distal end opposite the proximal end. The electrosurgical electrode also includes a cutting-electrode portion extending from the proximal end to the distal end, a coagulating-electrode portion extending from the proximal end to the distal end, and an insulator between the cutting-electrode portion and the coagulating-electrode portion. The cutting-electrode portion is configured for cutting tissue using the electrosurgical energy received from the electrosurgical tool, and the coagulating-electrode portion is configured for coagulating tissue using the electrosurgical energy received from the electrosurgical tool.

The electrosurgical tool includes a housing having a distal end and proximal end, at least one electrical conductor at the proximal end and configured to couple to an electrosurgical generator, a receptacle at the distal end and configured to couple to the proximal end of the electrosurgical electrode, and at least one user input device configured to select between a cutting mode of operation and a coagulation mode of operation. In the cutting mode of operation, the electrosurgical tool supplies the electrosurgical energy from the at least one electrical conductor to the cutting-electrode portion of the electrosurgical electrode and not the coagulating-electrode portion of the electrosurgical electrode. In the coagulation mode of operation, the electrosurgical tool supplies the electrosurgical energy from the at least one electrical conductor to at least the coagulating-electrode portion of the electrosurgical electrode.

In another example, a method of making an electrosurgical electrode for an electrosurgical tool includes forming a cutting-electrode portion, forming a coagulating-electrode portion, positioning an insulator between cutting-electrode portion and the coagulating-electrode portion, and coupling the cutting-electrode portion to the coagulating-electrode portion with the insulator between the cutting-electrode portion and the coagulating-electrode portion. The cutting-electrode portion is configured for cutting tissue using electrosurgical energy received from an electrosurgical tool. The coagulating-electrode portion is configured for coagulating tissue using the electrosurgical energy received from the electrosurgical tool.

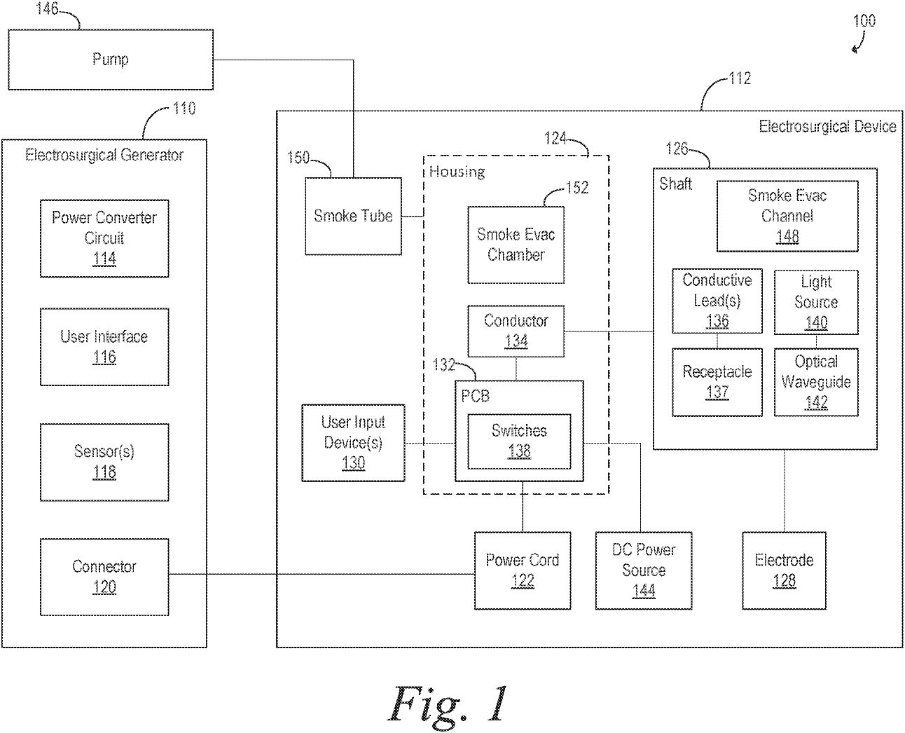

Referring now to FIG. 1, an electrosurgical system 100 is shown according to an example. As shown in FIG. 1, the electrosurgical system 100 includes an electrosurgical generator 110 and an electrosurgical tool 112. In general, the electrosurgical generator 110 can generate electrosurgical energy that is suitable for performing electrosurgery on a patient. For instance, the electrosurgical generator 110 can include a power converter circuit 114 that can convert a grid power to electrosurgical energy such as, for example, a radio frequency (RF) output power. As an example, the power converter circuit 114 can include one or more electrical components (e.g., one or more transformers) that can control a voltage, a current, and/or a frequency of the electrosurgical energy.

Within examples, the electrosurgical generator 110 can include a user interface 116 that can receive one or more inputs from a user and/or provide one or more outputs to the user. As examples, the user interface 116 can include one or more buttons, one or more switches, one or more dials, one or more keypads, one or more touchscreens, and/or one or more display screens.

In an example, the user interface 116 can be operable to select a mode of operation from among a plurality of modes of operation for the electrosurgical generator 110. As examples, the modes of operation can include a cutting mode, a coagulating mode, an ablating mode, and/or a sealing mode. Combinations of these waveforms can also be formed to create blended modes. In one implementation, the modes of operation can correspond to respective waveforms for the electrosurgical energy. As such, in this implementation, the electrosurgical generator 110 can generate the electrosurgical energy with a waveform selected from a plurality of waveforms based, at least in part, on the mode of operation selected using the user interface 116.

The electrosurgical generator 110 can also include one or more sensors 118 that can sense one or more conditions related to the electrosurgical energy and/or the target tissue. As examples, the sensor(s) 118 can include one or more current sensors, one or more voltage sensors, one or more temperature sensors and/or one or more bioimpedance sensors. Within examples, the electrosurgical generator 110 can additionally or alternatively generate the electrosurgical energy with an amount of electrosurgical energy (e.g., an electrical power) and/or a waveform selected from among the plurality of waveforms based on one or more parameters related to the condition(s) sensed by the sensor(s) 118.

In one example, the electrosurgical energy can have a frequency that is greater than approximately 100 kilohertz (kHz) to reduce (or avoid) stimulating a muscle and/or a nerve near the target tissue. In another example, the electrosurgical energy can have a frequency that is between approximately 300 kHz and approximately 500 kHz.

In FIG. 1, the electrosurgical generator 110 also includes a connector 120 that can facilitate coupling the electrosurgical generator 110 to the electrosurgical tool 112. For example, the electrosurgical tool 112 can include a power cord 122 having a plug, which can be coupled to a socket of the connector 120 of the electrosurgical generator 110. In this arrangement, the electrosurgical generator 110 can supply the electrosurgical energy to the electrosurgical tool 112 via the coupling between the connector 120 of the electrosurgical generator 110 and the power cord 122 of the electrosurgical tool 112.

As shown in FIG. 1, the electrosurgical tool 112 can include a housing 124 defining an interior chamber, a shaft 126 extending in a distal direction from the housing 124, and an electrosurgical electrode 128 coupled to the shaft 126. In general, the housing 124 can be configured to facilitate a user gripping and manipulating the electrosurgical tool 112 while performing electrosurgery. For example, the housing 124 can have a shape and/or a size that can facilitate a user performing electrosurgery by manipulating the electrosurgical tool 112 using a single hand. In one implementation, the housing 124 can have a shape and/or a size that facilitates the user holding the electrosurgical tool 112 in a writing utensil gripping manner (e.g., the electrosurgical tool 112 can be an electrosurgical pencil).

Additionally, for example, the housing 124 can be constructed from one or more materials that are electrical insulators (e.g., a plastic material). This can facilitate insulating the user from the electrosurgical energy flowing through the electrosurgical tool 112 while performing the electrosurgery.

In some implementations, the shaft 126 can be fixedly coupled to the housing 124. In other implementations, the shaft 126 can be telescopically moveable relative to the housing 124. For example, the shaft 126 can be telescopically moveable in an interior bore defined by the housing 124 to extend the shaft 126 in the distal direction and retract the shaft 126 in a proximal direction relative to the housing 124 (e.g., movable along a longitudinal axis of the electrosurgical tool 112). As noted above, the electrosurgical electrode 128 is coupled to the shaft 126 and, thus, the electrosurgical electrode 128 moves together with the shaft 126 relative to the housing 124. This can provide for adjusting a length of the electrosurgical tool 112, which can facilitate performing electrosurgery at a plurality of different depths within tissue (e.g., due to different anatomical shapes and/or sizes of patients) and/or at a plurality of different angles.

In some examples, the shaft 126 can additionally or alternatively be rotatable about an axis of rotation that is parallel to the longitudinal axis of the electrosurgical tool 112. In another example, the electrode 128 can be additionally or alternatively rotatable relative to the shaft 126. Rotating the shaft 126 and/or the electrosurgical electrode 128 relative to the housing 124 can facilitate adjusting an angle of the electrosurgical electrode 128 relative to one or more user input device(s) 130 of the electrosurgical tool 112. In this arrangement, a user can comfortably grip the housing 124 in a position in which their fingers can comfortably operate the user input device(s) 130 while the electrosurgical electrode 128 is set at a rotational position selected from among a plurality of rotational positions relative to the housing 124 based on, for example, a size and/or a shape of a surgical site in which the user is operating.

The user input device(s) 130 can select between the modes of operation of the electrosurgical tool 112 and/or the electrosurgical generator 110. For instance, in one implementation, the user input device(s) 130 can be configured to select between a cutting mode of operation and a coagulation mode of operation. Responsive to actuation of the user input device(s) 130 of the electrosurgical tool 112, the electrosurgical tool 112 can (i) receive the electrosurgical energy with a level of power and/or a waveform corresponding to the mode of operation selected via the user input device(s) 130 and (ii) supply the electrosurgical energy to the electrosurgical electrode 128.

In FIG. 1, the electrosurgical tool 112 includes a plurality of electrical components that facilitate supplying the electrosurgical energy, which the electrosurgical tool 112 receives from the electrosurgical generator 110, to the electrosurgical electrode 128. For example, the electrosurgical tool 112 can include a printed circuit board 132 (e.g., a flexible printed circuit board), a housing conductor 134, one or more conductive leads 136, and/or a receptacle 137 that can provide a circuit for conducting the electrosurgical energy from the power cord 122 to the electrosurgical electrode 128. One or more of the electrical components can be positioned in the internal chamber defined by the housing 124.

Within examples, the user input device(s) 130 can include one or more buttons on an exterior surface of the housing 124. Each button of the user input device(s) 130 can be operable to actuate a respective one of a plurality of switches 138 of the printed circuit board 132. In general, the switches 138 and/or the printed circuit board 132 are operable to control a supply of the electrosurgical energy from the electrosurgical generator 110 to the electrosurgical electrode 128. For instance, in one implementation, when each button is operated (e.g., depressed), the respective switch 138 associated with the button can be actuated to cause the printed circuit board 132 to transmit a signal to the electrosurgical generator 110 and cause the electrosurgical generator 110 to responsively supply the electrosurgical energy with a level of power and/or a waveform corresponding to a mode of operation associated with the button. In another implementation, operating the button and thereby actuating the respective switch 138 associated with the button can close the switch 138 to complete a circuit to the electrosurgical generator 110 to cause the electrosurgical generator 110 to responsively supply the electrosurgical energy with a level of power and/or a waveform corresponding to a mode of operation associated with the button. In some examples of this implementation, the printed circuit board 132 can be omitted.

In both example implementations, the electrosurgical energy supplied by the electrosurgical generator 110 can be supplied from (i) the power cord 122, the printed circuit board 132, and/or the switches 138 to (ii) the electrosurgical electrode 128 by the housing conductor 134 and the conductive lead(s) 136. As such, as shown in FIG. 1, the printed circuit board 132 can be coupled to the power cord 122, the housing conductor 134 can be coupled to the printed circuit board 132 and the conductive lead(s) 136, and the conductive lead(s) 136 can be coupled to the electrosurgical electrode 128 (e.g., via the receptacle 137). In this arrangement, the housing conductor 134 can conduct the electrosurgical energy (supplied to the housing conductor 134 via the printed circuit board 132) to the conductive lead(s) 136, the conductive lead(s) 136, and the receptacle 137 can conduct the electrosurgical energy to the electrosurgical electrode 128.

In general, the housing conductor 134 can include one or more conductive elements that provide an electrically conductive bus for supplying the electrosurgical energy to the electrosurgical electrode 128. In one example, the housing conductor 134 can be formed in a helical shape. In this arrangement, the housing conductor 134 can be compressible and expandable such that the housing conductor 134 can accommodate the shaft 126 telescopically moving into and/or out of the housing 124 to retract and/or extend, respectively, the electrosurgical electrode 128 relative to the housing 124. In another example, the conductive lead(s) 136 can include one or more wires. In another example, the conductive lead(s) 136 can include one or more conductive traces formed by, for instance, screen printing, sputtering, electroplating, conductive paint and/or laser ablation.

Within examples, the conductive lead(s) 136 can extend from the housing conductor 134 to the electrosurgical electrode 128. In one example, the conductive lead(s) 136 can include one or more wires. In another example, the conductive lead(s) 136 can include one or more conductive traces formed by, for instance, screen printing, sputtering, electroplating, conductive paint and/or laser ablation. The conductive lead(s) 136 can be disposed in an internal conduit of the shaft 126 and an exterior surface of the shaft 126 can be formed of an electrically insulating material. This can help reduce (or prevent) loss of the electrosurgical energy prior to the electrosurgical electrode 128.

The receptacle 137 can couple the electrosurgical electrode 128 to the electrosurgical tool 112. As an example, the receptacle 137 and the electrosurgical electrode 128 can be configured to couple to each other by friction-fit. Accordingly, the receptacle 137 and the electrosurgical electrode 128 can have respective sizes and/or respective shapes that provide for a friction-fit coupling between the receptacle 137 and the electrosurgical electrode 128 when the electrosurgical electrode 128 is inserted in the receptacle 137. This can allow for the electrosurgical electrode 128 to be releasably coupled to the electrosurgical tool 112, which can facilitate an interchangeability of a plurality of the electrosurgical electrodes 128 with the electrosurgical tool 112. The receptacle 137 and electrosurgical electrode 128 can be mechanically keyed to ensure the correct electrical connections are made. In other examples, the electrosurgical electrode 128 can be coupled to the receptacle 137 by another type of releasable coupling (e.g., a threaded coupling) or a non-releasable coupling (e.g., via welding and/or soldering).

Within examples, the receptacle 137 can also include a conductor that can electrically couple the electrosurgical electrode 128 to the electrosurgical energy supplied to the electrosurgical tool 112 by the electrosurgical generator 110. For instance, the receptacle 137 can be electrically coupled to the conductive lead(s) 136 (e.g., by a conductive material).

As shown in FIG. 1, the electrosurgical tool 112 can additionally include a light source 140 that is configured to emit light. In the example of FIG. 1, the light source 140 can optically coupled to an optical waveguide 142, which is configured to receive the light emitted by the light source 140 and transmit the light in a distal direction toward a surgical site to illuminate the surgical site while performing electrosurgery using the electrosurgical electrode 128. Within examples, the optical waveguide 142 can transmit the light in the distal direction via total internal reflection. For instance, the optical waveguide can include a cladding and/or an air gap on an exterior surface of the optical waveguide 142. In some implementations, the optical waveguide 142 can be formed as a single, monolithic structure. In another example, the electrosurgical tool 112 can omit the optical waveguide 142 and instead emit the light from the light source 140 directly to the surgical field without transmitting the light through the optical waveguide 142.

In FIG. 1, the light source 140 is coupled to the shaft 126. As such, the light source 140 can also move telescopically with the shaft 126 relative to the housing 124. However, in other examples, the light source 140 can be coupled to the housing 124. As examples, the light source 140 can include one or more light emitting diodes (LEDs), organic light emitting diodes (OLEDs), optical fibers, non-fiber optic waveguides, and/or lenses.

The optical waveguide 142 can be at a distal end of the shaft 126. In some examples, the electrosurgical electrode 128 can extend from a central portion of the optical waveguide 142. As such, the optical waveguide 142 can circumferentially surround the electrosurgical electrode 128 to emit the light distally around all sides of the electrosurgical electrode 128. This can help to mitigate shadows and provide greater uniformity of illumination in all rotational alignments of the shaft 126 relative to the housing 124 and/or the electrosurgical tool 112 relative to the target tissue.

In implementations that include the light source 140, the user input device(s) 130, the printed circuit board 132, the switches 138, the housing conductor 134, and/or the conductive lead(s) 136 can additionally supply an electrical power from a direct current (DC) power source 144 to the light source 140. In one example, the DC power source 144 can include a battery disposed in the housing 124 and/or the plug of the power cord 122. Although the electrosurgical tool 112 includes the DC power source 144 in FIG. 1, the DC power source 144 can be separate and distinct from the electrosurgical tool 112 in other examples. For instance, in another example, the electrosurgical generator 110 can include the DC power source 144.

Additionally, in implementations that include the light source 140, the user input device(s) 130 can be operable to cause the light source 140 to emit the light. In one example, the user input device(s) 130 can include a button that independently controls the light source 140 separate from the button(s) that control the electrosurgical operational modes of the electrosurgical tool 112. In another example, the user input device(s) 130 and the printed circuit board 132 can be configured such that operation of the button(s) that control the electrosurgical operational mode simultaneously control operation of the light source 140 (e.g., the light source 140 can be automatically actuated to emit light when a button is operated to apply the electrosurgical energy at the electrosurgical electrode 128).

As shown in FIG. 1, responsive to operation of the user input device(s) 130 to actuate the light source 140, the DC power source 144 can supply the electrical power (e.g., a DC voltage) to the light source 140 via the printed circuit board 132, the housing conductor 134, and/or the conductive lead(s) 136. In this implementation, one or more of the conductive elements of the housing conductor 134 can be configured to supply the electrical power from the DC power source 144 to the light source 140 and/or return the electrical power from the light source 140 to the DC power source 144. Accordingly, the housing conductor 134 can additionally or alternatively assist in providing electrical communication between the DC power source 144 and the light source 140 as the shaft 126 and the light source 140 telescopically move relative to the housing 124.

As noted above, the electrosurgical tool 112 can additionally include features that provide for evacuating surgical smoke from a target tissue to a location external to the surgical site. Surgical smoke is a by-product of various surgical procedures. For example, during surgical procedures, surgical smoke may be generated as a by-product of electrosurgical units (ESU), lasers, electrocautery devices, ultrasonic devices, and/or other powered surgical instruments (e.g., bones saws and/or drills). In some instances, the surgical smoke may contain toxic gases and/or biological products that result from a destruction of tissue. Additionally, the surgical smoke may contain an unpleasant odor. For these and other reasons, many guidelines indicate that exposure of surgical personnel to surgical smoke should be reduced or minimized.

To reduce (or minimize) exposure to surgical smoke, a smoke evacuation system may be used during the surgical procedure. In general, the smoke evacuation system may include a pump 146 that can generate sufficient suction and/or vacuum pressure to draw the surgical smoke away from the surgical site. In some implementations, the smoke evacuation system may be coupled to an exhaust system (e.g., an in-wall exhaust system) that exhausts the surgical smoke out of an operating room. In other implementations, the smoke evacuation system may filter air containing the surgical smoke and return the air to the operating room. Within examples, the pump 146 and the electrosurgical generator 110 can be provided as separate devices or integrated in a single device (e.g., in a common housing).

As shown in FIG. 1, the shaft 126 can include a smoke evacuation channel 148 at a distal end of the shaft 126. In an example, the smoke evacuation channel 148 can extend circumferentially around the optical waveguide 142 at the distal end of the shaft 126. The smoke evacuation channel 148 can also include a smoke inlet that extends circumferentially around the optical waveguide 142 at the distal end of the shaft 126. In this arrangement, the smoke inlet of the smoke evacuation channel can help to receive surgical smoke into the smoke evacuation channel 148 in all rotational alignments of the shaft 126 relative to the housing 124 and/or the electrosurgical tool 112 relative to the target tissue. However, in another example, the smoke evacuation channel 148 can include one or more smoke inlets that do not extend circumferentially around the optical waveguide 142 and/or the electrosurgical electrode 128.

In some implementations, the smoke evacuation channel 148 and the optical waveguide 142 can be coaxial. For instance, the smoke evacuation channel 148 and the optical waveguide 142 can each have a longitudinal axis that is aligned with a central axis of the shaft 126. In other implementations, the smoke evacuation channel 148 and the optical waveguide 142 can have respective longitudinal axes that are offset relative to each such that the smoke evacuation channel 148 and the optical waveguide 142 are not coaxial.

In an example, the smoke evacuation channel 148 can include an outer tube that is separated from the optical waveguide 142 by an air gap. For instance, the shaft 126 can include a plurality of standoffs that extend between the optical waveguide 142 and the outer tube of the smoke evacuation channel 148 to provide the air gap between the outer tube and the optical waveguide 142. In one implementation, the optical waveguide 142 can include the standoffs such that the optical waveguide 142 and the standoffs are formed as a single, monolithic structure. In another implementation, the standoffs can be formed as a single, monolithic structure with the outer tube of the smoke evacuation channel 148. In another implementation, the standoffs can be separate from the outer tube of the smoke evacuation channel 148 and the optical waveguide 142.

In an example, the smoke evacuation channel 148 of the shaft 126 defines a first portion of a smoke flow path, and the interior chamber of the housing 124 defines a second portion of a smoke flow path. In this arrangement, the surgical smoke can be received from the surgical site into the smoke evacuation channel 148 of the shaft 126, and flow proximally along the smoke evacuation channel 148 to the interior chamber of the housing 124. In the interior chamber of the housing 124, the smoke can further flow to a smoke tube 150 that is coupled to a proximal end of the housing 124 and configured to convey smoke from the housing 124 to the pump 146.

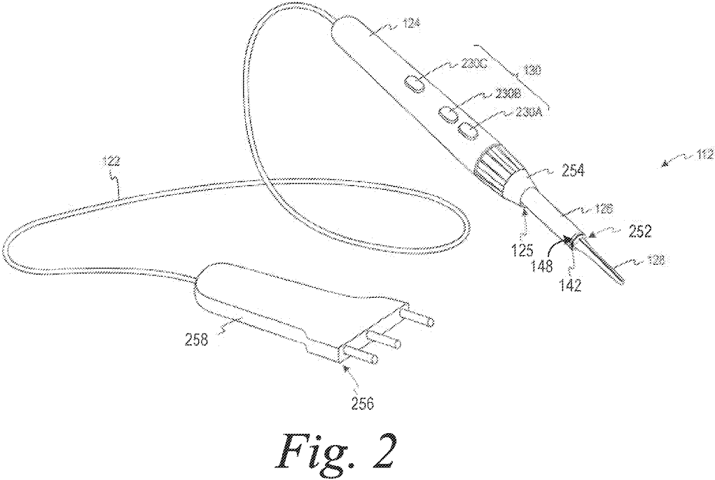

Referring now to FIG. 2, a perspective view of an implementation of the electrosurgical tool 112 is shown according to an example. As shown in FIG. 2, the electrosurgical tool 112 includes the housing 124, the shaft 126 telescopically moveable in the interior chamber of the housing 124, and the electrosurgical electrode 128 coupled to the shaft 126. However, as described above, the shaft 126 can be fixedly coupled to the housing 124 such that the shaft 126 is not moveable relative to the housing 124 in other examples.

Additionally, in FIG. 2, the optical waveguide 142 is at a distal end 252 of the shaft 126. In this arrangement, the optical waveguide 142 can telescopically move with the shaft 126 relative to the housing 124. In FIG. 2, the optical waveguide 142 extends around the electrosurgical electrode 128. This can help to emit the light in a relatively uniform manner by reducing (or preventing) shadows due to an orientation of the optical waveguide 142 and the electrosurgical electrode 128 relative to the surgical site. However, in other examples, the optical waveguide 142 may not extend entirely around the electrosurgical electrode 128 at the distal end 252 of the shaft 126, and/or the optical waveguide 142 can be at a different position on the shaft 126 and/or the housing 124.

In some examples, the electrosurgical tool 112 can include a collar 254 at a proximal end of the housing 124. The collar 254 can be rotatable relative to the housing 124 to increase and/or decrease friction between an outer surface of the shaft 126 and an inner surface of the collar 254. In this way, the collar 254 to allow and/or inhibit axial telescopic movement and/or rotational movement of the shaft 126 relative to the housing 124.

As shown in FIG. 2, the electrosurgical tool 112 includes the power cord 122. At a proximal end 256 of the power cord 122, the power cord 122 includes a plug 258 configured to couple to the connector 120 of the electrosurgical generator 110. A distal end of the power cord 122 is coupled to the printed circuit board 132 in the interior cavity of the housing 124. In this arrangement, the power cord 122 extends proximally from the housing 124 to the plug 258.

Additionally, as shown in FIG. 2, the user input device(s) 130 include a first button 230A, a second button 230B, and a third button 230C on an exterior surface of the housing 124. In one implementation, the first button 230A can be actuated to operate the electrosurgical tool 112 in a cutting mode of operation, the second button 230B can be actuated to operate the electrosurgical tool 112 in a coagulation mode of operation, and the third button 230C can be actuated to operate the light source 140 (i.e., to cause the light source 140 to emit light or cease emitting light). As described above, the user input device(s) 130 can be configured differently in other examples. For instance, the electrosurgical tool 112 can be operable in a lesser quantity of modes of operation, a greater quantity of modes of operation, and/or different types of modes of operation in other examples (e.g., such as the example modes of operation described above). Additionally, for instance, the at least one user input device 130 can additionally or alternatively include the user interface 116 of the electrosurgical generator 110 and/or another external device (e.g., a footswitch) for operating the electrosurgical tool 112 in one or more modes of operation.

Within examples, the electrosurgical electrode 128 can provide for selectively applying the electrosurgical energy to different portions of the electrosurgical electrode 128 based on the mode of operation in which the electrosurgical tool 112 is operated. The different portions of the electrosurgical electrode 128 can have a plurality of different sizes and/or a plurality of different shapes that can help to enhance one or more properties of the electrosurgical energy applied to the target tissue. In this way, the electrosurgical electrode 128 can enhance and/or improve operational performance of the electrosurgical tool 112 relative to conventional electrosurgical tools that conduct the electrosurgical energy through an entirety of the electrosurgical electrode 128 for all modes of operation.

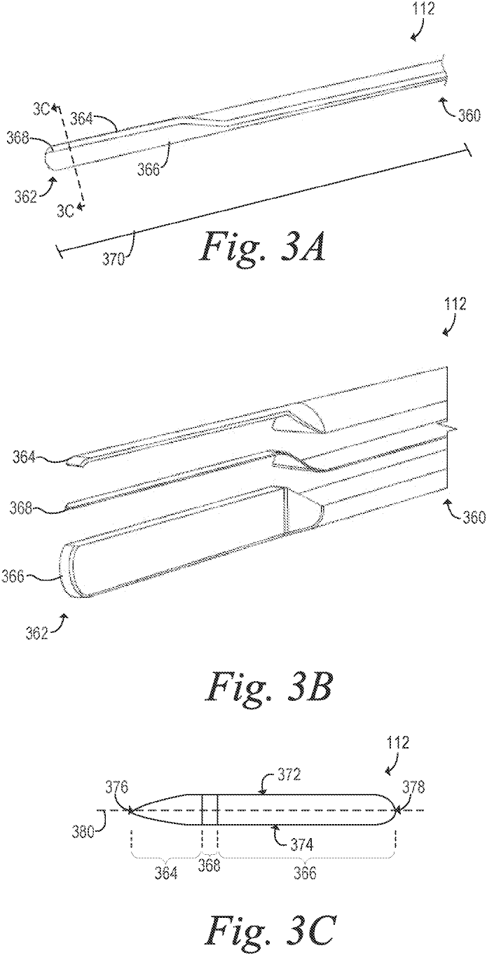

FIGS. 3A-3C depict the electrosurgical electrode 128 according to an example. In particular, FIG. 3A depicts a partial perspective view of the electrosurgical electrode 128, FIG. 3B depicts an exploded view of the electrosurgical electrode 128, and FIG. 3C depicts a cross-sectional view of the electrosurgical electrode 128 through a line 3C in FIG. 3A.

As shown in FIGS. 3A-3C, the electrosurgical electrode 128 includes a proximal end 360 configured to receive electrosurgical energy from an electrosurgical tool 112, and a distal end 362 opposite the proximal end 360. The proximal end 360 can receive electrosurgical energy from the electrosurgical tool 112, as described in further detail below. The distal end 362 can define a working end, which is configured for cutting and coagulating tissue using the electrosurgical energy.

As shown in FIGS. 3A-3C, the electrosurgical electrode 128 also includes a cutting-electrode portion 364 extending from the proximal end 360 to the distal end 362, a coagulating-electrode portion 366 extending from the proximal end 360 to the distal end 362, and an insulator 368 between the cutting-electrode portion 364 and the coagulating-electrode portion 366. The cutting-electrode portion 364 and the coagulating-electrode portion 366 can include a conductive material (e.g., stainless steel) for conducting the electrosurgical energy received from the electrosurgical tool 112 at the proximal end 360 to distal end 362. As described in further detail below, the cutting-electrode portion 364 and the coagulating-electrode portion 366 can define separate portions of the electrosurgical electrode 128 that can be selectively energized to apply the electrosurgical energy to the target tissue according to a selected mode of operation.

The insulator 368 can separate the cutting-electrode portion 364 from the coagulating-electrode portion 366 over an entire length 370 of the electrosurgical electrode 128 in a direction that is parallel to a longitudinal axis of the electrosurgical electrode 128. The insulator 368 can include an electrically insulating material that can inhibit conducting the electrosurgical energy between the cutting-electrode portion 364 and the coagulating-electrode portion 366. In this way, the insulator 368 can facilitate conducting the electrosurgical energy through one of the cutting-electrode portion 364 or the coagulating-electrode portion 366 with a negligible or no electrosurgical energy being conducted through the other one of the cutting-electrode portion 364 or the coagulating-electrode portion 366.

For example, the insulator 368 can include a material selected from a group consisting of a plastic, a ceramic, and an enamel. Additionally, for example, the insulator 368 can have a thickness that is equal to or greater than a thickness of the cutting-electrode portion 364 and/or a thickness of the coagulating-electrode portion 366. In one example, the insulator 368 can have a width that between approximately 0.1 millimeters (mm) and approximately 0.5 mm. This can help to electrically insulate the cutting-electrode portion 364 and the coagulating-electrode portion 366 from each other. Although it is advantageous to separate the cutting-electrode portion 364 and the coagulating-electrode portion 366 by the insulator 368, the cutting-electrode portion 364 and the coagulating-electrode portion 366 can be separated by an air gap in another example.

In FIGS. 3A-3C, the cutting-electrode portion 364 is configured for cutting tissue using the electrosurgical energy received from the electrosurgical tool 112, and the coagulating-electrode portion 366 is configured for coagulating tissue using the electrosurgical energy received from the electrosurgical tool 112. For instance, a surface area of the cutting-electrode portion 364 can be smaller than a surface area of the coagulating-electrode portion 366. This can help to achieve a relatively greater density of electrosurgical energy when applying the electrosurgical energy to the cutting-electrode portion 364, and a relatively less density of electrosurgical energy when applying the electrosurgical energy to the coagulating-electrode portion 366. As a relatively greater density of electrosurgical energy can help to enhance performance during a cutting operation and a relatively lesser density of electrosurgical energy can help to enhance performance during a coagulating operation, the relative sizes of the cutting-electrode portion 364 and the coagulating-electrode portion 366 can help to improve performance of the electrosurgical tool 112.

In one example, the surface area of the cutting-electrode portion 364 is approximately 5 percent to approximately 50 percent of the surface area of the coagulating-electrode portion 366. In another example, the surface area of the cutting-electrode portion 364 is approximately 5 percent to approximately 35 percent of the surface area of the coagulating-electrode portion 366. In another example, the surface area of the cutting-electrode portion 364 is approximately 10 percent to approximately 25 percent of the surface area of the coagulating-electrode portion 366.

The cutting-electrode portion 364 and the coagulating-electrode portion 366 can additionally or alternatively have different shapes to improve the cutting operation using the cutting-electrode portion 364 and/or the coagulating operation using the coagulating-electrode portion 366. For instance, as shown in FIG. 3C, the electrosurgical electrode 128 can include a first side 372 extending between the proximal end 360 and the distal end 362, a second side 374 extending between the proximal end 360 and the distal end 362, a first edge 376 at a first lateral interface between the first side 372 and the second side 374, and a second edge 378 at a second lateral interface between the first side 372 and the second side 374. The first side 372 and the second side 374 are on opposing sides of an intermediate plane 380 extending through the first edge 376 and the second edge 378. The cutting-electrode portion 364 includes the first edge 376 and the coagulating-electrode portion 366 includes the second edge 378.

As shown in FIG. 3C, the first edge 376 is thinner and sharper than the second edge 378. This can additionally or alternatively help to achieve a relatively greater density of electrosurgical energy when applying the electrosurgical energy to the cutting-electrode portion 364, and a relatively less density of electrosurgical energy when applying the electrosurgical energy to the coagulating-electrode portion 366. As such, the first edge 376 of the cutting-electrode portion 364 can help to achieve relatively better performance than the second edge 378 of the coagulating-electrode portion 366 for a cutting operation, and the second edge 378 of the coagulating-electrode portion 366 can help to achieve relatively better performance than the first edge 376 of the cutting-electrode portion 364 for a coagulating operation.

As noted above, the insulator 368 separates and inhibits electrical coupling between the cutting-electrode portion 364 and the coagulating-electrode portion 366. In an example, a longitudinal axis extends from the proximal end 360 toward the distal end 362 and, along the longitudinal axis, the cutting-electrode portion 364 and the coagulating-electrode portion 366 are separated by approximately 0.1 mm to approximately 0.5 mm. This separation distance can help to inhibit electrical coupling between the cutting-electrode portion 364 and the coagulating-electrode portion 366.

Within examples, the cutting-electrode portion 364 and/or the coagulating-electrode portion 366 can be covered in a non-stick material (e.g., a material having a relatively low coefficient of friction) including at least one material selected from silicone, siloxane and Teflon. This can help to mitigate tissue adhering to the electrosurgical electrode 128. When tissue adheres to an electrosurgical electrode, the tissue may change the effective size and/or shape of the electrode. As such, tissue adherence may impair making relatively narrow and precise incisions and, thus, negatively impact a quality and/or a speed of the electrosurgical procedure. However, cutting-electrode portion 364 and the coagulating-electrode portion 366 with the non-stick material having a relatively low coefficient of friction can help to mitigate tissue adhering to the electrosurgical electrode 128 as the electrosurgical electrode 128 moves through the target tissue during electrosurgery and, thus, improve the quality and/or speed of the electrosurgical procedure.

In some examples, the cutting-electrode portion 364 and/or the coagulating-electrode portion 366 can be additionally or alternatively covered in an insulation material such as, for instance, a polymeric material and/or a fluorocarbon material (e.g., polytetrafluoroethylene (PTFE)). In one example, the layer of insulation material can be a coating (e.g., an insulating enamel).

As described above, the proximal end 360 of the electrosurgical electrode 128 can receive electrosurgical energy from the electrosurgical tool 112. For instance, as described above, the electrosurgical electrode 128 can be coupled to the receptacle 137 by a releasable coupling (e.g., a friction-fit or a threaded coupling) or a non-releasable coupling (e.g., via welding and/or soldering). In an implementation, the receptacle 137 is configured to removably couple to the proximal end 360 of the electrosurgical electrode 128 by a friction-fit coupling.

Within examples, the electrosurgical tool 112 and the electrosurgical electrode 128 selectively applying the electrosurgical energy to (i) only the cutting-electrode portion 364, (ii) only the coagulating-electrode portion 366, and/or (iii) both the cutting-electrode portion 364 and the coagulating-electrode portion 366. This can be achieved by providing the electrosurgical tool 112 with a plurality of electrical circuits for coupling the cutting-electrode portion 364 and the coagulating-electrode portion 366 to the electrosurgical generator 110.

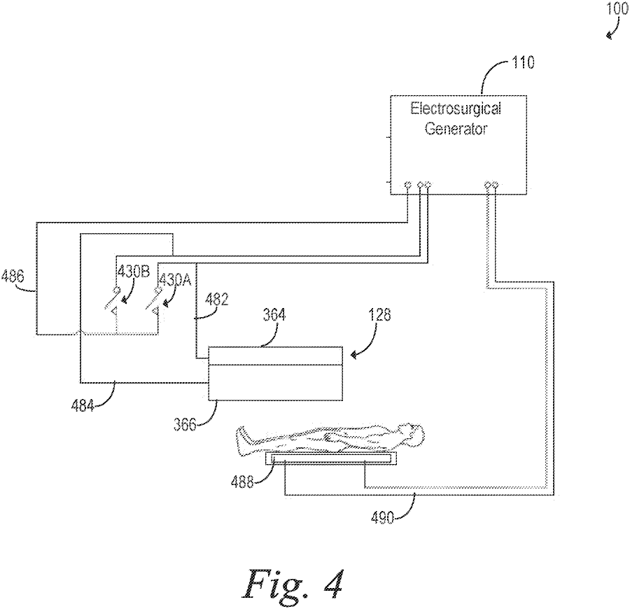

FIG. 4 illustrates a schematic circuit diagram for the electrosurgical system 100 according to an example. In example shown in FIG. 4, the electrosurgical energy is conducted through the cutting-electrode portion 364 and not through the coagulation-electrode portion 366 when the electrosurgical tool 112 is operated in a cutting operation, and the electrosurgical energy is conducted through the coagulating-electrode portion 366 and not through the cutting-electrode portion 364 when the electrosurgical tool 112 is operated in a coagulating operation.

For instance, in FIG. 4, the at least one user input device 130 includes a first user input device 430A that is operable to cause the electrosurgical generator 110 to supply the electrosurgical energy to the cutting-electrode portion 364. Additionally, in FIG. 4, the at least one user input device 130 includes a second user input device 430B that is operable to cause the electrosurgical generator 110 to supply the electrosurgical energy to the coagulating-electrode portion 366. The first user input device 430A can include a first switch that is operable by the first button 230A (shown in FIG. 2) to operate the electrosurgical tool 112 in the cutting mode of operation, and the second user input device 430B can include a second switch that is operable by the second button 230B (shown in FIG. 2) to operate the electrosurgical tool 112 in a coagulation mode of operation.

As described above, the electrosurgical tool 112 can include at least one electrical conductor selected from the power cord 122, the housing conductor 134, and the conductive lead(s) 136 for electrically coupling the electrosurgical electrode 128 to the electrosurgical generator 110. In FIG. 4, the at least one electrical conductor includes a first conductor 482 that is configured to couple the cutting-electrode portion 364 to the electrosurgical generator 110, and a second conductor 484 that is configured to couple the coagulating-electrode portion 366 to the electrosurgical generator 110.

The at least one electrical conductor also includes a third conductor 486 that electrically couples the electrosurgical generator 110 to the first switch of the first user input device 430A. The first switch of the first user input device 430A is actuatable between an open state and a closed state. In the open state of the first switch, the third conductor 486 is decoupled from the first conductor 482 and the cutting-electrode portion 364. In the closed state of the first switch, the third conductor 486 is coupled to the first conductor 482 and the cutting-electrode portion 364.

In FIG. 4, the third conductor 486 also electrically couples the electrosurgical generator 110 to the second switch of the second user input device 430B. The second switch of the second user input device 430B is actuatable between an open state and a closed state. In the open state of the second switch, the third conductor 486 is decoupled from the second conductor 484 and the coagulating-electrode portion 366. In the closed state of the second switch, the third conductor 486 is coupled to the second conductor 484 and the cutting-electrode portion 364.

In this arrangement, when the first switch of the first user input device 430A is actuated to the closed state and the second switch of the second user input device 430B remains in the open state, the electrosurgical generator 110 supplies the electrosurgical energy to the cutting-electrode portion 364 and not the coagulating-electrode portion 366. Whereas, when the second switch of the second user input device 430B is actuated to the closed state and the first switch of the first user input device 430A remains in the open state, the electrosurgical generator 110 supplies the electrosurgical energy to the coagulating-electrode portion 366 and not the cutting-electrode portion 364.

Accordingly, in the cutting mode of operation, the electrosurgical tool 112 supplies the electrosurgical energy from the at least one electrical conductor to the cutting-electrode portion 364 of the electrosurgical electrode 128 and not the coagulating-electrode portion 366 of the electrosurgical electrode 128. Whereas, in the coagulation mode of operation, the electrosurgical tool 112 supplies the electrosurgical energy from the at least one electrical conductor to the coagulating-electrode portion 366 of the electrosurgical electrode 128 and not the cutting-electrode portion 364 of the electrosurgical electrode 128.

As shown in FIG. 4, the electrosurgical system 100 can also include a dispersive electrode 488 and a plurality of return conductors 490 coupling the dispersive electrode 488 to the electrosurgical generator 110. The dispersive electrode 488 and the return conductors 490 can return electric current from the patient to the electrosurgical generator 110.

In the example shown in FIG. 4, the electrosurgical energy is conducted through the cutting-electrode portion 364 and not through the coagulation-electrode portion 366 when the electrosurgical tool 112 is operated in the cutting mode of operation, and the electrosurgical energy is conducted through the coagulating-electrode portion 366 and not through the cutting-electrode portion 364 when the electrosurgical tool 112 is operated in a coagulating mode of operation. However, in another example, the electrosurgical energy can be conducted through both the coagulating-electrode portion 366 and the cutting-electrode portion 364 when the electrosurgical tool 112 is operated in the coagulating mode of operation.

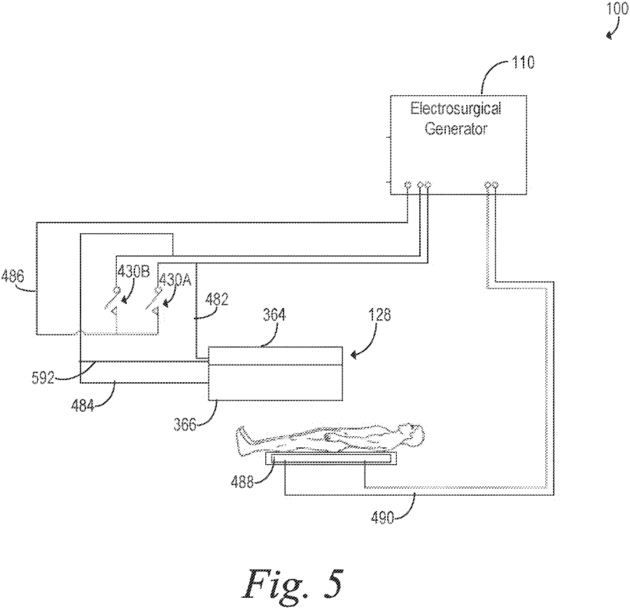

FIG. 5 illustrates a schematic circuit diagram for the electrosurgical system 100 according to one such example. The schematic circuit diagram of FIG. 5 is substantially similar or identical to the schematic circuit diagram of FIG. 4, except the electrosurgical system 100 further includes a fourth conductor 592 that provides for electrically coupling the cutting-electrode portion 364 to the electrosurgical generator 110 when the second switch of the second user input device 430B is actuated to the closed state.

Accordingly, in the arrangement shown in FIG. 5, (i) the electrosurgical energy is conducted through the cutting-electrode portion 364 and not through the coagulation-electrode portion 366 when the electrosurgical tool 112 is operated in the cutting mode of operation, and (ii) the electrosurgical energy is conducted through the coagulating-electrode portion 366 and the cutting-electrode portion 364 when the electrosurgical tool 112 is operated in a coagulating mode of operation. This can help to increase a surface area of the electrosurgical electrode 128 that conducts the electrosurgical energy and, thus, decrease a density of the electrosurgical energy when using the electrosurgical tool 112 in the coagulating mode of operation.

As described above, providing the insulator 368 between the cutting-electrode portion 364 and the coagulating-electrode portion 366 can help to inhibit the electrosurgical energy from being conducted between the cutting-electrode portion 364 and the coagulating-electrode portion 366. This can allow for selectively activating different portions of the electrosurgical electrode 128 according to different modes of operation.

In some examples, the insulator 368 between the cutting-electrode portion 364 and the coagulating-electrode portion 366 can additionally or alternatively provide for the electrosurgical electrode 128 acting as a sensor that can sense one or more conditions related to an electrosurgical operation. For instance, in an example, the electrosurgical generator 110 can use the electrical properties of the cutting-electrode portion 364 and the coagulating-electrode portion 366 to measure an impedance of a target tissue. Referring to FIG. 4, in the example of user input device 430A being actuated and the cutting-electrode portion 364 being active, a high impedance, though not open circuit, on the second conductor 484 can be used to allow a relatively small current to pass from the cutting-electrode portion 364 to the coagulating-electrode portion 366. To bypass the insulator 368 between the cutting-electrode portion 364 and the coagulating-electrode portion 366, the current has to pass through a portion of the tissue immediately adjacent to the cutting-electrode portion 364 and the coagulating-electrode portion 366. This relatively small current will pass through the tissue and return to the electrosurgical generator 110.

The electrosurgical generator 110 can then use this current to measure an electrical characteristic such as, for example, an impedance, a voltage, a capacitance, and/or an inductance. The electrosurgical generator 110 can determine, based on the measured electrical characteristic, a characteristic of a tissue (e.g., the impedance of the tissue, a water content of the tissue, a density of the tissue, a fat content of the tissue, and/or a temperature of the tissue) to which the electrosurgical electrode 128 has applied the electrosurgical energy. Additionally or alternatively, the electrosurgical generator 110 can use the measured electrical characteristic to set and/or modify a power and/or a waveform of the electrosurgical energy supplied to the electrosurgical tool 112. In this way, the electrosurgical electrode 128 can beneficially provide a measurement functionality of the tissue while the electrosurgical device 112 is being used.

In another example, as before with the cutting-electrode portion 364 activated, rather than using a high impedance on the second conductor 484, the electrosurgical generator 110 can intermittently create a circuit with the cutting-electrode portion 364 and the coagulating-electrode portion 366 acting as two poles such that a path of least resistance is from the cutting-electrode portion 364 through the immediately adjacent tissue, bypassing the insulating layer 368 and into the coagulating-electrode portion 366. The electrosurgical generator 110 can use this circuit to measure the electrical characteristic and determine, based on the electrical characteristic, the characteristic of the tissue as described above. This measurement can be made in the order of milliseconds such that cutting performance is not noticeably affected.

In another example, the electrical characteristic between the activated portion(s) of the electrosurgical electrode 128 (e.g., the portions selected from the cutting-electrode portion 364 and/or the coagulating-electrode portion 366 that are being supplied with the electrosurgical energy) and the return electrode 488 is measured within the electrosurgical generator 110. For instance, the impedance of the tissue immediately adjacent to the electrode 128 can be measured in addition to the impedance between the activated portion(s) of the electrosurgical electrode 128 and the return electrode 488. Over multiple measurements the impedance between the activated portion(s) of the electrosurgical electrode 128 through the patient to the return electrode 488 can average out such that a discernable change in impedance is specifically associated with the tissue immediately adjacent to the activated portion(s) of the electrosurgical electrode 128.

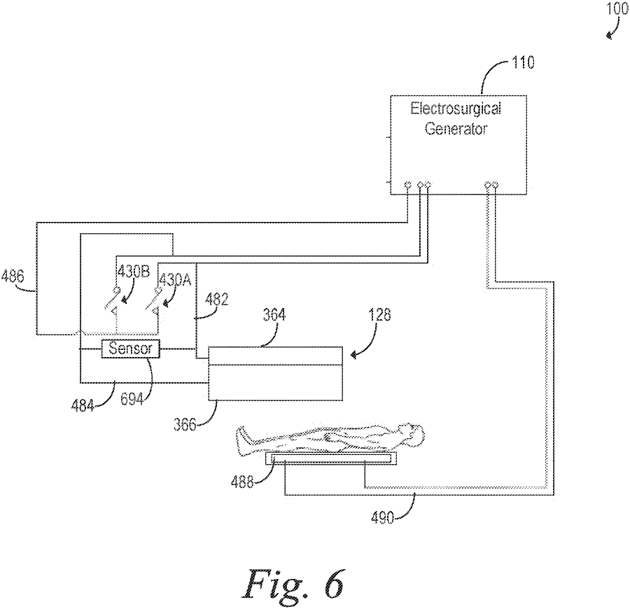

FIG. 6 illustrates a schematic circuit diagram for the electrosurgical system 100 according to one such example. The schematic circuit diagram of FIG. 6 is substantially similar or identical to the schematic circuit diagrams of FIG. 4, except the electrosurgical system 100 further includes a sensor 694 that provides for measuring the electrical characteristic (e.g., an impedance, a voltage, a phase, a capacitance, and/or an inductance) across the cutting-electrode portion 364 and the coagulating-electrode portion 366. In some implementations, the sensor 694 can be located in the electrosurgical generator 110. In other implementations, the sensor 694 can be located in the electrosurgical tool 112. In either implementation, the sensor 694 can generate a sensor signal indicative of the electrical characteristic measured by the sensor 694. The sensor 694 can further transmit the sensor signal to the electrosurgical generator 110, which can determine the characteristic of the tissue based on the sensor signal, and responsively set and/or modify the power and/or a waveform of the electrosurgical energy supplied to the electrosurgical tool 112 as described above.

As described above, the electrosurgical generator 110 can perform various operations including, for example, measuring the electrical characteristic, determining the characteristic of the tissue, setting the waveform and/or the power of the electrosurgical energy, and/or modifying the waveform and/or the power of the electrosurgical energy. Within examples, the electrosurgical generator 110 can include one or more controller that are configured to carry out at least these operations. The controller(s) can be implemented using hardware, software, and/or firmware. For instance, the controller(s) can include one or more processors and a non-transitory computer readable medium (e.g., volatile and/or non-volatile memory) that stores machine language instructions or other executable instructions. The instructions, when executed by the one or more processors, cause the electrosurgical generator 110 to carry out the various operations described herein. The controller(s), thus, can receive data and store the data in the memory as well.

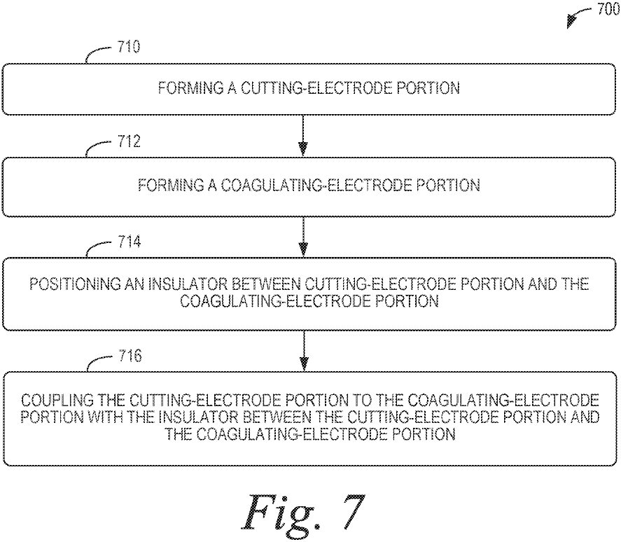

Referring now to FIG. 7, a flowchart is shown for a process 700 of making an electrosurgical electrode for an electrosurgical tool according to an example. As shown in FIG. 7, the process 700 includes forming a cutting-electrode portion at block 710, forming a coagulating-electrode portion at block 712, positioning an insulator between cutting-electrode portion and the coagulating-electrode portion at block 714, and coupling the cutting-electrode portion to the coagulating-electrode portion with the insulator between the cutting-electrode portion and the coagulating-electrode portion at block 716. The cutting-electrode portion is configured for cutting tissue using electrosurgical energy received from an electrosurgical tool. The coagulating-electrode portion is configured for coagulating tissue using the electrosurgical energy received from the electrosurgical tool.

The description of the different advantageous arrangements has been presented for purposes of illustration and description, and is not intended to be exhaustive or limited to the examples in the form disclosed. Many modifications and variations will be apparent to those of ordinary skill in the art. Further, different advantageous examples may describe different advantages as compared to other advantageous examples. The example or examples selected are chosen and described in order to explain the principles of the examples, the practical application, and to enable others of ordinary skill in the art to understand the disclosure for various examples with various modifications as are suited to the particular use contemplated.

What is claimed is:

1. An electrosurgical electrode for an electrosurgical tool, the electrosurgical electrode comprising:
a proximal end configured to receive electrosurgical energy from an electrosurgical tool;
a distal end opposite the proximal end;
a cutting-electrode portion extending from the proximal end to the distal end, wherein the cutting-electrode portion is configured for cutting tissue using the electrosurgical energy received from the electrosurgical tool;
a coagulating-electrode portion extending from the proximal end to the distal end, wherein the coagulating-electrode portion is configured for coagulating tissue using the electrosurgical energy received from the electrosurgical tool; and
an insulator between the cutting-electrode portion and the coagulating-electrode portion.

2. The electrosurgical electrode of claim 1, wherein a surface area of the cutting-electrode portion is smaller than a surface area of the coagulating-electrode portion.

3. The electrosurgical electrode of claim 2, wherein a surface area of the cutting-electrode portion is approximately 5 percent to approximately 50 percent of a surface area of the coagulating-electrode portion.

4. The electrosurgical electrode of claim 1, wherein a longitudinal axis extends from the proximal end toward the distal end, and
wherein, along the longitudinal axis, the cutting-electrode portion and the coagulating-electrode portion are separated by approximately 0.1 - 0.5 millimeters (mm).

5. The electrosurgical electrode of claim 1, wherein the cutting-electrode portion is separated from the coagulating-electrode portion over an entire length of the electrosurgical electrode between the proximal end and the distal end.

6. The electrosurgical electrode of claim 1, further comprising:
a first side extending between the proximal end and the distal end;
a second side extending between the proximal end and the distal end;
a first edge at a first lateral interface between the first side and the second side; and
a second edge at a second lateral interface between the first side and the second side,
wherein the first side and the second side are on opposing sides of an intermediate plane extending through the first edge and the second edge, and
wherein the cutting-electrode portion includes the first edge, the coagulating-electrode portion includes the second edge, and the first edge is thinner than the second edge.

7. The electrosurgical electrode of claim 1, wherein the insulator comprises a material selected from a group consisting of a plastic, a ceramic, and an enamel.

8. The electrosurgical electrode of claim 1, wherein the cutting-electrode portion and the coagulating-electrode portion are covered in a non-stick material comprising at least one material selected from silicone, siloxane and Teflon.

9. An electrosurgical system comprising:
an electrosurgical electrode comprising:
a proximal end configured to receive electrosurgical energy from an electrosurgical tool;
a distal end opposite the proximal end;
a cutting-electrode portion extending from the proximal end to the distal end, wherein the cutting-electrode portion is configured for cutting tissue using the electrosurgical energy received from the electrosurgical tool;
a coagulating-electrode portion extending from the proximal end to the distal end, wherein the coagulating-electrode portion is configured for coagulating tissue using the electrosurgical energy received from the electrosurgical tool; and
an insulator between the cutting-electrode portion and the coagulating-electrode portion; and
an electrosurgical tool comprising:
a housing having a distal end and proximal end,
at least one electrical conductor configured to couple the electrosurgical electrode to an electrosurgical generator,
a receptacle at the distal end configured to couple to the proximal end of the electrosurgical electrode, and
at least one user input device configured to select between a cutting mode of operation and a coagulation mode of operation,
wherein, in the cutting mode of operation, the electrosurgical tool supplies the electrosurgical energy from the at least one electrical conductor to the cutting-electrode portion of the electrosurgical electrode and not the coagulating-electrode portion of the electrosurgical electrode, and
wherein, in the coagulation mode of operation, the electrosurgical tool supplies the electrosurgical energy from the at least one electrical conductor to at least the coagulating-electrode portion of the electrosurgical electrode.

10. The electrosurgical system of claim 9, wherein the at least one user input device comprises:
a first input device that is operable to cause the electrosurgical generator to supply the electrosurgical energy to the cutting-electrode portion; and
a second input device that is operable to cause the electrosurgical generator to supply the electrosurgical energy to the coagulating-electrode portion.

11. The electrosurgical system of claim 9, wherein the at least one electrical conductor comprises:
a first conductor that is configured to couple the cutting-electrode portion to the electrosurgical generator; and
a second conductor that is configured to couple the coagulating-electrode portion to the electrosurgical generator.

12. The electrosurgical system of claim 9, wherein the receptacle is configured to removably couple to the proximal end of the electrosurgical electrode by a friction-fit coupling.

13. The electrosurgical system of claim 9, wherein a surface area of the cutting-electrode portion is smaller than a surface area of the coagulating-electrode portion.

14. The electrosurgical system of claim 13, wherein a surface area of the cutting-electrode portion is approximately 5 percent to approximately 35 percent of a surface area of the coagulating-electrode portion.

15. The electrosurgical system of claim 9, wherein a longitudinal axis extends from the proximal end toward the distal end, and
wherein, along the longitudinal axis, the cutting-electrode portion and the coagulating-electrode portion are separated by approximately 0.1 millimeters (mm) to approximately 0.5 mm.

16. The electrosurgical system of claim 9, wherein the cutting-electrode portion is separated from the coagulating-electrode portion over an entire length of the electrosurgical electrode between the proximal end and the distal end.

17. The electrosurgical system of claim 9, further comprising:
a first side extending between the proximal end and the distal end; a second side extending between the proximal end and the distal end;
a first edge at a first lateral interface between the first side and the second side; and
a second edge at a second lateral interface between the first side and the second side,
wherein the first side and the second side are on opposing sides of an intermediate plane extending through the first edge and the second edge, and
wherein the cutting-electrode portion includes the first edge, the coagulating-electrode portion includes the second edge, and the first edge is thinner than the second edge.

18. The electrosurgical system of claim 9, wherein the insulator comprises a material selected from a group consisting of a plastic, a ceramic, and an enamel.

19. The electrosurgical system of claim 9, wherein the cutting-electrode portion and the coagulating-electrode portion are covered in a non-stick material comprising at least one material selected from silicone, siloxane and Teflon.

20. A method of making an electrosurgical electrode for an electrosurgical tool, the method comprising:
forming a cutting-electrode portion;
forming a coagulating-electrode portion;
positioning an insulator between cutting-electrode portion and the coagulating-electrode portion; and
coupling the cutting-electrode portion to the coagulating-electrode portion with the insulator between the cutting-electrode portion and the coagulating-electrode portion,
wherein the cutting-electrode portion is configured for cutting tissue using electrosurgical energy received from an electrosurgical tool, and
wherein the coagulating-electrode portion is configured for coagulating tissue using the electrosurgical energy received from the electrosurgical tool.

21. An electrosurgical system, comprising:
an electrosurgical electrode comprising:
a proximal end configured to receive electrosurgical energy from an electrosurgical tool;
a distal end opposite the proximal end;
a cutting-electrode portion extending from the proximal end to the distal end, wherein the cutting-electrode portion is configured for cutting tissue using the electrosurgical energy received from the electrosurgical tool;
a coagulating-electrode portion extending from the proximal end to the distal end, wherein the coagulating-electrode portion is configured for coagulating tissue using the electrosurgical energy received from the electrosurgical tool; and
an insulator between the cutting-electrode portion and the coagulating-electrode portion;
an electrosurgical tool comprising:
a housing having a distal end and proximal end,
at least one electrical conductor configured to couple the electrosurgical electrode to an electrosurgical generator,
a receptacle at the distal end configured to couple to the proximal end of the electrosurgical electrode, and
at least one user input device configured to select between a cutting mode of operation and a coagulation mode of operation,
wherein, in the cutting mode of operation, the electrosurgical tool supplies the electrosurgical energy from the at least one electrical conductor to the cutting-electrode portion of the electrosurgical electrode and not the coagulating-electrode portion of the electrosurgical electrode, and
wherein, in the coagulation mode of operation, the electrosurgical tool supplies the electrosurgical energy from the at least one electrical conductor to at least the coagulating-electrode portion of the electrosurgical electrode; and
an electrosurgical generator coupled to the electrosurgical tool, wherein the electrosurgical generator is configured to supply electrosurgical energy to the electrosurgical tool,
wherein the electrosurgical generator is configured to:
measure at least one of an electrical characteristic between the cutting-electrode portion and the coagulating-electrode portion, and
determine, based on the at least one of the electrical characteristic, a characteristic of a tissue to which the electrosurgical electrode has applied the electrosurgical energy.

22. The electrosurgical system of claim 21, wherein the electrosurgical generator is configured to modify at least one of a power or a waveform of the electrosurgical energy supplied to the electrosurgical tool based on the at least one of the resistance or the voltage measured between the cutting-electrode portion and the coagulating-electrode portion.

23. The electrosurgical system of claim 21, wherein the electrical characteristic is at least one electrical characteristic selected from a group consisting of: an impedance, a voltage, a capacitance, and an inductance.

24. The electrosurgical system of claim 21, wherein the characteristic of the tissue is at least one characteristic selected from a group consisting of: an impedance of the tissue, a water content of the tissue, a density of the tissue, a fat content of the tissue, and a temperature of the tissue.
